(19) United States
(12) Patent Application Publication
(10) Pub. No.: US 2015/0297534 A1
(43) Pub. Date: Oct. 22, 2015

(54) METHODS AND COMPOSITIONS FOR THE MEDIATION OF NSAID-INDUCED REACTIONS

(71) Applicant: NuBioPharma, LLC, Sanford, NC (US)

(72) Inventors: Inderjit Kumar Dev, Durham, NC (US); Ajay Kumar Ajmani, Chapel Hill, NC (US)

(73) Assignee: NuBioPharma, LLC, Sanford, NC (US)

(21) Appl. No.: 14/688,398

(22) Filed: Apr. 16, 2015

Related U.S. Application Data
(60) Provisional application No. 61/981,468, filed on Apr. 18, 2014.

(51) Int. Cl. A61K 31/09; A61K 47/24; A61K 31/19; A61K 31/192

(52) U.S. Cl. CPC A61K 31/09; A61K 31/192; A61K 47/24; A61K 31/19

(57) ABSTRACT

Administration of an NSAID with at least one dual inhibitor of mPGES-1 and NF-κB plus at least one zwitterionic phospholipid rests in a lessening of side effects and the ability to administer smaller doses of an NSAID without loss of effectiveness.

This application claims priority of U.S. provisional application No. 61/981,468 filed on Apr. 18, 2014 and is incorporated in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to the administration of NSAIDS. In particular, it relates to the administration of NSAIDS with reduced adverse effects.

2. Description of Related Art

Nonsteroidal anti-inflammatory drugs, usually abbreviated to NSAIDs, are a class of drugs that provides analgesic and antipyretic (fever-reducing) effects, and, in higher doses, anti-inflammatory effects. The chronic use of NSAIDs is associated with adverse drug reaction (ADRs) of either gastrointestinal (GI), cardiovascular, and renal types. GI toxicity in many patients manifests as dyspepsia, ulcers, or bleeding. It has been estimated that ulcers occur in 15-30% of regular NSAID users. Mortality and morbidity associated with NSAID GI toxicity is also substantial. It has been reported that as a result of ulcer perforations and bleeding 16,000 NSAID users die each year in the US, and more than 100,000 are hospitalized each year in the US. COX-2 selective inhibitors, also referred to as coxibs, cause significantly less GI damage, while achieving anti-inflammatory and analgesic efficacy. A family of coxibs, including Celebrex, Vioxx, and Bextra, were previously approved as GI-safer NSAIDs. However, the coxibs also show significantly increased risk of causing serious cardiovascular side effects, and all but Celebrex (which is a less selective COX-2 inhibitor) have been withdrawn from the market in the US.

The toxicity of NSAIDs is dose related and lower doses of many NSAIDs, including ibuprofen, aspirin, naproxen, diclofenac, ketoprofen, methyl salicylate and choline salicylate, are approved for non-prescription over the counter (OTC) use in the US based on a favorable safety profile from the post-marketing data from many years of human use. The same NSAIDs are also available at higher doses by prescription in US for anti-inflammatory use for chronic diseases. In general, the OTC dose of an NSAID is safer than the prescription dose of the same NSAID. For example, low-dose analgesic OTC ibuprofen (1200 mg/day) is relatively safe and has no obvious major health issues associated with it, as it has a much lower probability of adverse GI events, renal and cardiovascular events. In contrast, daily use of a high dose of anti-inflammatory Ibuprofen (2400 mg/day) in arthritis patients for 24 weeks results in a very high incidence (22.9%) of gastric ulcers and is also associated with higher incidence of cardiovascular events and kidney toxicity. It is clear that using the lowest effective dose of NSAID for the shortest duration will mediate the NSAID-induced reactions in humans.

One strategy employs dietary ingredients in an attempt to increase the therapeutic effectiveness and potency of NSAIDs. The inclusion of these dietary agents is particularly attractive because of our long-standing exposure to them, their relative lack of toxicity, and encouraging validation from centuries of traditional use. However, identifying which, if any, ingredients are effective in a synergistic manner is not yet known.

One theory is that NSAIDs bind to phospholipids, notably phosphatidylcholine (PC) in the GI lining and compromise the lining's acid-repelling properties. Over time, the disruption of the GI lining can lead to clinically significant, and sometimes life-threatening damage, such as ulceration, bleeding and perforation. In support of this hypothesis, it has been shown that, in animal models, phospholipids from soy when complexed with an NSAID, reduced gastrointestinal irritating effects and enhanced antipyretic, analgesic, and anti-inflammatory activity. Aspirin-phoshatidylcholine (PC) complex reduced the aspirin-induced damage to the gastric mucosa in healthy human volunteers. In a clinical trial of osteoarthritic patients, a complex of ibuprofen and phosphatidylcholine had some encouraging ulcer preventive activity in older subjects, but very weak or no activity in younger people. Aspirin-PC complex is now approved for sale in the US. NSAIDs that are associated with phospholipids, such as phosphatidylcholine (“PC-NSAIDs”) are now considered new drugs that have fewer gastrointestinal side effects than regular NSAIDs. Clearly this approach has promise and new drugs in this field will be a welcome addition to NSAID therapy, as they are still urgently needed.

Prostaglandin E₂ (PGE₂) is a main mediator of fever, pain, and inflammation. Its biosynthesis from arachidonic acid involves several enzymes including Cyclooxygenase (COX)-1 or COX-2 and PGE₂ synthases (PGES). Inhibition of both COX-1 and COX-2 by nonsteroidal anti-inflammatory drugs (NSAIDs) like ibuprofen, naproxen, and aspirin and selective inhibition of COX-2 by coxibs like Celebrex and Vioxx reduces PGE₂ levels and leads to relief from pain, fever and inflammation.

Curcumin is the active ingredient of turmeric, with a long history of medicinal use in India and China, especially to treat inflammation.

### BRIEF SUMMARY OF THE INVENTION

This present invention relates to the discovery that the administration of a combination of at least one dual inhibitor of mPGES-1 and NF-κB, at least one zwitterionic phospholipids, with at least one NSAID act synergistically, more so than would be predicted from any one in combination with the NSAID. The administration in the same formulation, or at the same time, is synergistic in that it leads to an unpredicted enhancement of the inhibition of PGE₂, a main mediator of fever, pain, and inflammation. This observation can lead to the ability to administer a smaller dose of NSAID with the same effectiveness as the larger dose, or a larger dose of an NSAID without increased side effects, and can lead to an overall improvement in the safety profile of this regimen.

Accordingly, in one embodiment of the present invention, there is a composition for improving the analgesic, antipyretic and anti-inflammatory effects of a non-steroidal anti-inflammatory drug (NSAID) in a mammal in need of NSAID therapy comprising:
a) at least one dual inhibitor of microsomal prostaglandin E₂ synthase-1 (mpges-1) and NF-κB;
b) at least one zwitterionic phospholipid; and
c) a therapeutically effective amount of one or more NSAID.

And, in another embodiment, there is a method of treating a mammal in need of at least one of an analgesic, antipyretic and anti-inflammatory treatment using a (NSAID) in a mammal in need of NSAID therapy, comprising administering to the mammal an effective amount of a composition comprising:
a) at least one dual inhibitor of microsomal prostaglandin E₂ synthase-1 (mpges-1) and NF-κB;
b) at least one zwitterionic phospholipid; and
c) a therapeutically effective amount of one or more NSAID.

### BRIEF DESCRIPTION OF THE DRAWINGS

FIG. 1 is a chart of prostanoid formation in whole blood.

FIG. 2 is a chart showing the synergy with curcumin.

FIG. 3 is a chart of Stimulation of TNF-α Levels After Ibuprofen 400 mg Dose in Human Volunteer.

FIG. 4 is a chart showing curcumin suppresses TNF-alpha.

FIG. 5 is a chart showing the inhibition of IL01 beta by curcumin in a whole blood assay.

### DETAILED DESCRIPTION OF THE INVENTION

While this invention is susceptible to embodiment in many different forms, there is shown in the drawings, and will herein be described in detail, specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### DEFINITIONS

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term “comprising” could be separated into one or more claims using “consisting” or “consisting of” claim language and is so intended.

References throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or” as used herein, is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein, the term “mammal” is defined as any class of warm-blooded higher vertebrates, and that includes humans.

As used herein, the term “NSAIDs” denotes drugs like ibuprofen, naproxen, and aspirin that inhibit both COX-1 or COX-2 enzymes, or drugs like Celebrex and Vioxx that are selective inhibitors of COX-2.

As used herein, the term “curcumin” denotes either pure curcumin or a mixture of 3 curcuminoids (80% curcumin, 15% Demethoxycurcumin, 5% Bisdemethoxycurcumin).

As used herein, the term “PC-NSAIDs” denotes a non-covalently associated composition of a zwitterionic phospholipid and an NSAID i.e the zwitterionic phospholipid can be complexed with the NSAID.

As used herein, the term “phospholipid” refers any lipid or fatty acid having a covalently attached a phosphate group in the molecular structure.

As used herein, the term “mPGES-1 inhibitor” denotes any pharmaceutical agent having specific inhibitory activity for the microsomal prostaglandin E₂ synthase (mPGES)-1, one of the terminal enzymes of PGE₂ biosynthesis that is induced by inflammatory stimuli.

As used herein, the phrase “NF-κB inhibitor” denotes any pharmaceutical agent having specific inhibitory activity for NF-κB (nuclear factor kappa-light-chain-enhancer of activated B cells), a protein complex that controls transcription of DNA responsible for cytokine production and cell survival.

As used herein, various Interleukins are designated as (IL-1β, IL-6, IL-8 and IL-18), tumor necrosis factor-α (TNF-α), Interferon gamma-induced protein 10 (IP-10) monocyte chemotactic protein-1 (MCP-1), Macrophage inflammatory protein 1 alpha (MIP-1 alpha), regulated on activation normal T cell expressed and secreted (RANTES), and Inducible nitric oxide synthase (iNOS).

As used herein, prostanoids are a group of lipid compounds consisting of the prostaglandins (PGE₂, PGI₂, PGD₂, PGF_{2α}), the thromboxanes (TXA₂), and the prostacyclins (PGI₂. and 6-keto PGF_{1α}, is a stable degradation product of prostacyclin).

As used herein, the phrase “various matrix metalloproteinases (such as MMP-1, -3, -9 and -13)” refers to enzymes that are involved in the breakdown of extracellular matrix and, during tissue remodeling in normal physiological processes as well as in disease processes, such as arthritis, and tumor metastasis.

As used herein the term “neoplasm” refers to an abnormal growth of cells or tissue and is understood to include benign, i.e., non-cancerous growths, and malignant, i.e., cancerous growths. The term “neoplastic” means of or related to a neoplasm.

As used herein the term “agent” is understood to mean a substance that produces a desired effect in a tissue, system, animal, mammal, human, or other subject. It is also to be understood that an “agent” may be a single compound or a combination or composition of two or more compounds.

As used herein, the phrase “effective amount” means the amount of a drug or pharmaceutical agent that will elicit the biological or medical response of a tissue, system, animal or human that is being sought, for instance, by a researcher or clinician. Furthermore, the phrase “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The phrase also includes within its scope amounts effective to enhance normal physiological function.

As used herein, the phrase “reducing a therapeutic dose size” means that the amount or concentration of one NSAID is reduced by using the novel pharmaceutical combination of the invention by at least about 5%, 10%, 20%, 30%, 40%, 50%, 60%, 70%, 80%, 90% or 95% as compared to the amount or concentration of one NSAID in a single dosage, if administered alone for the treatment of the same disease or disorder in order to achieve the same effect.

As used herein, the phrase “Dual inhibitor of mPGES-1 and NF-κB” refers to compositions which inhibit both compositions with a single composition.

As recited above, the method and treatment combination of the present invention also includes at least one dual mPGES-1 and NF-κB inhibitor. Generally, any mPGES-1/NF-κB inhibitor that is any pharmaceutical agent having specific mPGES-1 and/or NF-κB inhibitor activity may be utilized in the present invention. Such mPGES-1/NF-κB inhibitors are described, for instance, in [Nam N H. Mini Rev Med Chem. 2006 August; 6(8):945-51; Chang, H. and Meuillet E. J. Future Med Chem. 2011; 3(15) 1909-1934; Oettl S K et al. PLoS One 2013 Oct. 9; 8(10): e76929; Schaible A M et al. Biochem Pharmacol. 2013 Aug. 15; 86(4):476-86; Bauer J et al. J Pharmacol Exp Ther. 2012 July; 342(1):169-76; Baumgartner L et al. J Nat Prod. 2011 Aug. 26; 74(8)1779-86] and are herein incorporated by reference to the extent of their disclosure of their mPGES-1 and NF-κB inhibitor activity and methods of preparing and using the same.

Natural mPGES-1/NF-κB inhibitors include curcumin from turmeric, epi-gallocatechin gallate from green tea, garcinol from Guttiferae species, myrtucommulone from myrtle, arzanol from Helichrysum italicum, β-boswellic acid, keto-β-boswellic acid, and acetyl-keto-β-boswellic acid (AKBA) from frankincense, 2-(2-hydroxy-4-methoxyphenyl)-5-(3-hydroxypropyl)benzofuran, (+)-conocarpan and other lignin derivatives from Krameria lappacea, diterpenes carnosol and carnosic acid from Salvia officinalis, physodic acid, imbricaric acid and perlatolic acid and other depsides and depsidones from Lichens, Embelin (2,5-dihydroxy-3-undecyl-1,4-benzoquinone) from Embelia Ribes seeds, acylphloroglucinol hyperforin from St. John's Wort as described in [Nam N H. Mini Rev Med Chem. 2006 August; 6(8):945-51; Chang, H. and Meuillet E. J. Future Med Chem. 2011; 3(15) 1909-1934; Oettl S K et al. PLoS One 2013 Oct. 9; 8(10): e76929; Schaible A M et al. Biochem Pharmacol. 2013 Aug. 15; 86(4):476-86; Bauer J et al. J Pharmacol Exp Ther. 2012 July; 342(1):169-76; Baumgartner L et al. J Nat Prod. 2011 Aug. 26; 74(8)1779-86] and are herein incorporated by reference.

In one embodiment, a dual mPGES-1/NF-κB inhibitor may be selected from boswellic acids and their derivatives, as described in patent applications WO2008058514 A1 and WO2009117987 A2, which are herein incorporated by reference to the extent of their disclosure of their mPGES-1 and/or NF-κB inhibitor activity and methods of preparing and using the same.

In one other embodiment, a dual mPGES-1 and NF-κB inhibitors may be selected from curcumin, demethoxycurcumin, bisdemethoxycurcumin and their mixtures, as described in patent application US 20070093457 which is herein incorporated by reference to the extent of its disclosure of their mPGES-1 and/or NF-κB inhibitor activity and methods of preparing and using the same.

The concentration of the dual inhibitor of mPGES-1 and NF-κB needed to achieve the therapeutic effect as compared to administering at least one NSAID alone, may be measured by testing several varying combinations of the dual inhibitor of mPGES-1 and NF-κB and the at least one NSAID. Any combination containing the dual inhibitor of mPGES-1 and NF-κB and the at least one NSAID, for which the NSAID concentration is low, yet maintaining therapeutic efficacy, as compared to concentrations of NSAID alone, in in vitro, in vivo and clinical tests, would be considered as the combination obtained by the method above.

As used herein the phrase “zwitterionic phospholipids” refers to a phospholipid having a proton acceptor in the molecular structure so that the phosphate group can bear a negative charge and the proton acceptor can bear a positive charge due to an intra-molecular acid-base reaction.

The novel pharmaceutical combination of the present invention also includes at least one zwitterionic; for example, those mentioned in U.S. Pat. Nos. 5,763,422 and 5,955,451, which are herein incorporated by reference to the extent of their methods of making and using the same.

Suitable examples of other zwitterionic phospholipids include, without limitation, phosphatidylcholines such as phosphatidyl choline (PC), dipalmitoylphosphatidylcholine (DPPC), other disaturated phosphatidylcholines, phosphatidylethanolamines, phosphatidylinositol, phosphatidylserines sphingomyelin or other ceramides, or various other zwitterionic phospholipids, phospholipid containing oils such as lecithin oils derived from soy beans, dimyristoylphosphatidylcholine, distearoylphosphatidylcholine, dilinoleoylphosphatidylcholine (DLL-PC), dipalmitoylphosphatidylcholine (DPPC), soy phophatidylchloine (Soy-PC or PC_{S}) and egg phosphatidycholine (Egg-PC or PC_{E}). In Egg-PC, which is a mixture of unsaturated phospholipids, R₁ primarily contains a saturated aliphatic substitution (e.g., palmitic or stearic acid), and R₂ is primarily an unsaturated aliphatic substitution (e.g., oleic or arachidonic acid). In Soy-PC, which in addition to the saturated phospholipids (palmitic acid and stearic acid) is a mixture of unsaturated phospholipids, [oleic acid, linoleic acid and linolenic acid]. The embodiments of zwitterionic phospholipid include, without limitation, dipalmitoyl phosphatidylcholine, phosphatidyl choline, or a mixture thereof.

As used herein the term “NSAID” refers to any Non-steroidal anti-inflammatory drug and is well known in the art, with optional pharmaceutically acceptable excipients in the fixed unit dosage form according to the present invention. Suitable NSAIDS include, without limitation: (a) propionic acid drugs including fenoprofen calcium, flurbiprofen, suprofen, benoxaprofen, Ibuprofen, ketoprofen, naproxen, and/or oxaprozin; (b) acetic acid drug including diclofenac sodium, diclofenac potassium, aceclofenac, etodolac, indoniethacin, ketorolac tromethamine, and/or ketorolac; (c) ketone drugs including nabumetone. sulindac. and/or tolmetin sodium: (d) fenamate drugs including meclofenamate sodium, and/or metenamic acid; (e) oxicam drugs piroxicam, lomoxicam and meloxicam; (f) salicylic acid drugs including diflunisal, aspirin, magnesium salicylate, bismuth subsalicylate, and/or other salicylate pharmaceutical agents; (g) pyrazolin acid drugs including oxyphenbutazone, and/or phenylbutazone; and (h) mixtures or combinations thereof. Suitable COX-2 inhibitors include, without limitation, celecoxib, rofecoxib, valdecoxib or mixtures and combinations thereof, a NO-releasing NSAID, a salt, a hydrate or an ester thereof, e.g. NO-releasing diclofenac or NO-releasing naproxen.

The PC, NSAID and the dual mPGES-1 and NF-κB inhibitor may be employed in combination in accordance with the invention by administration concomitantly in (1) a unitary pharmaceutical composition including all compositions (e.g. used of a PC-NSAID complex) or (2) separate pharmaceutical compositions each including one of the compounds. Alternatively, the combination may be administered separately in a sequential manner wherein, for example, the PC, then NSAID or dual mPGES-1 and NF-κB inhibitor is administered first and the other second. Such sequential administration may be close in time or remote in time.

Typically, the compositions may be administered as a desired salt. Salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from nitrogen on a substituent in a compound of the present invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

While it is possible that, for use in therapy, therapeutically effective amounts of a PC, NSAID and the dual mPGES-1 and NF-κB inhibitor, as well as salts, solvates and physiological functional derivatives thereof, may be administered as the raw chemical, it is possible to present the active ingredient as a pharmaceutical composition. Accordingly, the invention further provides pharmaceutical compositions, which include therapeutically effective amounts of a PC-NSAID and the dual mPGES-1 and NF-κB inhibitor and salts, solvates and physiological functional derivatives thereof, and one or more pharmaceutically acceptable carriers, diluents, or excipients. The compounds of the present invention and salts, solvates, and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention, there is also provided a process for the preparation of a pharmaceutical formulation including mixing a PC-NSAID complex and the dual mPGES-1 and NF-κB inhibitor or salts, solvates and physiological functional derivatives thereof, with one or more pharmaceutically acceptable carriers, diluents or excipients.

The compounds of the invention may be administered as prodrugs. Examples of prodrugs include phosphate prodrugs, such as dihydrogen or dialkyl (e.g. di-tert-butyl) phosphate prodrugs. Further examples of replacement groups in accordance with the foregoing examples and examples of other prodrug types may be found in the aforementioned references.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of an NSAID and/or mPGES-1 inhibitor, depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

The PC-NSAID and the dual mPGES-1 and NF-κB inhibitor combination may be administered by any appropriate route. Suitable routes include oral, rectal, nasal, topical (including buccal and sublingual), vaginal, and parenteral (including subcutaneous, intramuscular, intraveneous, intradermal, intrathecal, and epidural). It will be appreciated that the preferred route may vary with, for example, the condition of the recipient of the combination. It will also be appreciated that each of the agents may be administered by the same or different routes and that the PC, NSAID and the dual mPGES-1 and NF-κB inhibitors may be compounded together in a pharmaceutical composition/formulation.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic, pharmaceutically acceptable inert carrier, such as ethanol, glycerol, water and the like. Powders are prepared by comminuting the compound to a suitable fine size and mixing with a similarly comminuted pharmaceutical carrier such as an edible carbohydrate, as, for example, starch or mannitol. A flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be granulated, the powder mixture can be run through the tablet machine, and the result is imperfectly formed slugs broken into granules. The granules can be lubricated and be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base, as described above, and, optionally, with a binder such as carboxymethylcellulose, an alginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids, such as solutions, syrups and elixirs, can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers, such as ethoxylated isostearyl alcohols and polyoxyl ethylene sorbitol ethers, preservatives, flavor additives such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as, for example, by coating or embedding particulate material in polymers, wax or the like.

The agents for use according to the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Agents for use according to the present invention may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid, include a coarse powder having a particle size, for example, in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists that may be generated by means of various types of metered dose pressurized aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example, those suitable for oral administration may include flavoring agents.

As indicated, therapeutically effective amounts of the specific mPGES-1 and NF-κB inhibitor and an PC-NSAID are administered to a mammal. Typically, the therapeutically effective amount of one of the administered agents of the present invention will depend upon a number of factors including, for example, the age and weight of the mammal, the precise condition requiring treatment, the severity of the condition, the nature of the formulation, and the route of administration. Ultimately, the therapeutically effective amount will be at the discretion of the attendant physician or veterinarian.

Typically, the PC-NSAID and dual mPGES-1 and NF-κB inhibitors will be given in the range of 0.1 to 100 mg/kg body weight of recipient (mammal) per day and more usually in the range of 1 to 10 mg/kg body weight per day.

As noted above, the combinations of a PC-NSAID and dual mPGES-1 and NF-κB inhibitor described in the invention are useful because they exhibit synergistic inhibition of PGE2, a main mediator of fever, pain, and inflammation in healthy human volunteers, and also block the deleterious increase of inflammatory cytokines induced by NSAIDs. More particularly, the compounds of the invention are of use in the treatment of disorders for which an NSAID or an mPGES-1 inhibitor or an NF-κB inhibitor is indicated in an animal. Preferably, the animal is a mammal, more preferably a human.

The following examples are intended for illustration only and are not intended to limit the scope of the invention in any way.

### EXAMPLES

### Example 1

### Methods

As used herein the symbols and conventions used in these processes, schemes and examples are consistent with those used in the contemporary scientific literature, for example, the Journal of the American Chemical Society or the Journal of Biological Chemistry. Unless otherwise noted, all starting materials were obtained from commercial suppliers and used without further purification. Specifically, the following abbreviations may be used in the examples and throughout the specification: g (grams); mg (milligrams); L (liters); mL (milliliters); mu.L (microliters); M (molar); mM (millimolar); mu.M (micromolar); mol (moles); mmol (millimoles); RT (room temperature); min (minutes); h (hours); RP (reverse phase); HOAc (acetic acid); MeOH (methanol) HPLC (high pressure liquid chromatography); DMSO (dimethylsulfoxide); PBS phosphate buffered saline; and RPMI Roswell Park Memorial Institute.

Unless otherwise indicated, all temperatures are expressed in degree C. (degrees Centigrade). All reactions conducted under an inert atmosphere at room temperature unless otherwise noted.

Determination of PGE₂ and 6-keto PGF_{1α} Formation in Human Whole Blood: Peripheral blood from healthy adult volunteers, who had not received any medication for at least 2 weeks under informed consent, was obtained by venipuncture and collected in a vacutainer blood collection tube containing heparin. Aliquots of whole blood (0.8 ml) were mixed with aspirin (50 μM) and volume was adjusted to 1 ml with PBS containing 6 mM glucose. The blood was incubated with the indicated compounds for 5 mins. at room temperature; and then stimulated with lipopolysaccharide (10 μg/ml) for 18 h at 37° C. Prostanoid formation was stopped on ice and the samples were centrifuged (2300 g, 10 min, 4° C.), and 6-keto PGF_{1α} was quantified in the supernatant using High Sensitivity ELISA Kit for 6-keto PGF_{1α} according to the manufacturer's protocols (Calbiochem). PGE₂ was determined after solid-phase extraction, RP-HPLC and an ELISA according to the procedures of Koeberle et al. [J Pharmacol Exp Ther. 2008; 326(3):975-82].

Determination of Inflammatory Cytokines (IL-1β & TNF-α) in Human Whole Blood: Blood from healthy adult volunteers was obtained in vacutainer blood collection tubes containing heparin as described above. Aliquots of whole blood (0.2 ml) were diluted with serum-free medium (RPMI-SF) in 24-well culture plates and volume was adjusted to 1 ml. The blood was incubated with the indicated compounds for 5 minutes at room temperature, and then stimulated with lipopolysaccharide (10 μg/ml) for 6 h at 37° C. with 5% CO₂. Reaction was stopped on ice, the samples were centrifuged (2300 g, 10 mins., 4° C.), and of Inflammatory Cytokines IL-1β& TNF-α were estimated in the supernatant using High Sensitivity ELISA Kits according to the manufacturer's protocols (R&D Biosystems).

Human Healthy Volunteer Clinical Study: Volunteers with acute or chronic diseases, medical history of gastrointestinal ulcers or allergy to NSAIDs were not included. Volunteers were not permitted to receive any NSAIDs for 2 weeks before and throughout the duration of the study. The study was conducted in an open-label, crossover design with a seven-day washout period between each treatment. Volunteer 1 was given a single oral dose of 400 mg of ibuprofen (Baby Advil Suspension) with 240 mL of water. Administration of the study medication was supervised by the investigator to ensure that the drug had been swallowed. A Blood sample (6 ml), obtained via an indwelling catheter inserted into an arm vein, and was collected into a tube containing heparin immediately prior to an ibuprofen dosing and at the following times thereafter: 65, 105, and 115 mins. after dosing. An aliquot of blood was used for determination of PGE₂ and TNF-α after LPS stimulation as described above. Plasma was separated by centrifugation at 1000 g for 10 minutes at room temperature from the remaining blood for ibuprofen assay. After seven days (washout period), the same volunteer was given a single oral dose of 100 mg ibuprofen (Baby Advil Suspension) with 240 mL of water. A 6.0 mL blood sample for the determination of PGE₂ and TNF-α after LPS stimulation and for the estimation of plasma levels of ibuprofen were drawn into tubes through an indwelling cannula before dosing and at 75, 125, and 155 mins. after dosing.

Total plasma concentrations of ibuprofen were determined by high-performance liquid chromatography (HPLC.) system that consisted of a Model 510 pump, a Model 486 variable wavelength UV detector and a computer loaded with the Millenium software, all from Waters Associates (Milford, Mas). To 100 μl of plasma was added 100 μl of internal standard solution (naproxen 10 μg/ml in 65% methanol and 1% acetic acid) followed by 300 μl. Following vortex-mixing (30 s) and centrifugation (1000 g, 5 mins.), the supernatant was evaporated to dryness, and reconstituted in 100 μl of 65% methanol and 1% acetic acid immediately prior to injection onto a reversed phase column (ρBondaPak C18) eluted with methanol:1% acetic acid (65:35), pumped at 1.0 ml min. The column eluent was monitored at 210 nm. The height of the ibuprofen peak was divided by the height of the internal standard peak and this ratio was used to determine ibuprofen concentrations from a standard curve of ibuprofen.

Based on results from volunteer 1, a lower dose of ibuprofen and one sampling time that allowed ibuprofen and curcumin to enter the circulation was chosen for a 3-way crossover study. In this 3-way crossover study, volunteer 1 received single doses of 1) 50 mg ibuprofen 2) ibuprofen, curcumin or 3) ibuprofen, curcumin and PC; with a seven day washout period between the dosing of each drug or drug combinations. A Blood sample (6 ml), immediately prior to dosing and at 105 minutes after dosing was obtained as described above. Production of PGE₂, 6-keto PGF_{1α} and TNF-α after LPS stimulation and plasma concentrations were determined as described above.

In another 3-way crossover study, volunteer 2 received single doses of 1) 40 mg ibuprofen 2) ibuprofen, curcumin or 4) ibuprofen, curcumin and PC; with a seven day washout period between the dosing of each drug or drug combinations. A blood sample (6 ml), immediately prior to dosing and at 105 min after dosing was obtained as described above. Production of PGE₂, 6-keto PGF_{1α} and TNF-α after LPS stimulation and plasma concentrations were determined as described above.

Compounds of Example 2 are curcumin and ibuprofen. Curcumin was purchased from Sabinsa Corp. N.J. and contains 95% curcuminoids (80% curcumin, 15% Demethoxycurcumin, 5% Bisdemethoxycurcumin). Ibuprofen sodium salt was purchased from Sigma Biochemicals.

Compounds of Example 3 are the same as for Example 2.

Compounds of Example 4: Curcumin (25.0 g), ascorbyl palmitate (1.0 g), phosphatidylcholine (54 g), hydrogenated castor oil (2.0 g) and Polyvinyl Pyrrolidone (18.0 g) were suspended in 100 ml of absolute ethanol. The mixture was homogenized and heated at 60 deg C. The mixture was evaporated under a vacuum to remove ethanol. The dried mass was then pulverized in a mortar with a pestle to yield a yellow powder and 800 mg of this powder (containing 200 mg of curcumin and 400 mg of phosphatidylcholine) was dissolved in 240 ml of water and mixed with 50 mg of commercially available ibuprofen (Baby Advil) and used for dosing in the human volunteer in Example 4.

Compounds of Example 5 were the same as that for Example 4 except that 800 mg of the yellow powder (containing 200 mg of curcumin and 400 mg of phosphatidylcholine) was dissolved in 240 ml of water and mixed with 40 mg of commercially available ibuprofen (Baby Advil) and used for dosing in the human volunteer in Example 5.

Compounds of Example 6 were the same as that for Example 4 except that two higher doses (400 mg, 100 mg) of commercially available ibuprofen (Baby Advil) were also used for dosing in the human volunteer in Example 5.

Compounds of Example 7 were the same as described above for Example 2 and Example 4.

Synergistic interaction between compounds was analyzed by the sensitization method (Harvey, R. J., J. Theor. Biol. 74: 411-437, 1978). Briefly, sensitization is measured as the ratio between observed and expected inhibition of PGE₂. The expected level of activity (Aₑ) is calculated by A_{β}=1−((1−A₁)*(1−A₂)) (³) where A₁ and A₂ are the activities of drugs 1 and 2 alone at the concentration used in the combination (Harvey, R. J., J. Theor. Biol. 74: 411-437, 1978). A sensitization ratio (SR) of 1.0 suggests that the two inhibitors are acting independently, and a value above 1.0 indicates sensitization.

### Example 2

### mPGES-1 is the Molecular Target for Curcumin that Results in Inhibition of PGE₂

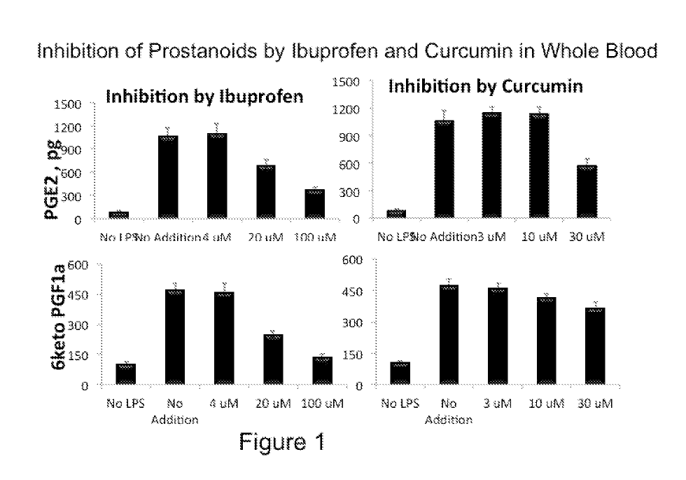

Koeberle A et al. [Mol Cancer Ther. 2009 August; 8(8):2348-55] taught us that mPGES-1 is the molecular target for curcumin. The present inventors also confirmed these findings. Whole human blood stimulated with lipopolysaccharide (10 mug/ml) was incubated with varied concentrations of ibuprofen and curcumin alone for 18 hours. Formation of two prostanoids PGE₂ and 6-keto PGF_{1α}, a stable metabolite of prostacyclin, a prostaglandin needed for cardiovascular function were determined by methods described earlier. An inhibitor of COX enzymes, ibuprofen inhibited the formation of both the prostanoids PGE₂ and 6-keto PGF_{1α}, to about the same extent (FIG. 1). As observed by Koeberle A et al. [Mol Cancer Ther. 2009 August; 8(8):2348-55] curcumin was a differential inhibitor of these two prostanoids, it inhibited PGE₂ more potently than 6-keto PGF_{1α} (FIG. 1). Koeberle A et al. [Mol Cancer Ther. 2009 August; 8(8):2348-55] has demonstrated that the differential inhibition of the prostanoids is predominantly due to inhibition of mPGES-1 by curcumin.

### Example 3

### Synergistic Interaction Between Curcumin and Ibuprofen is Mediated Through mPGES-1

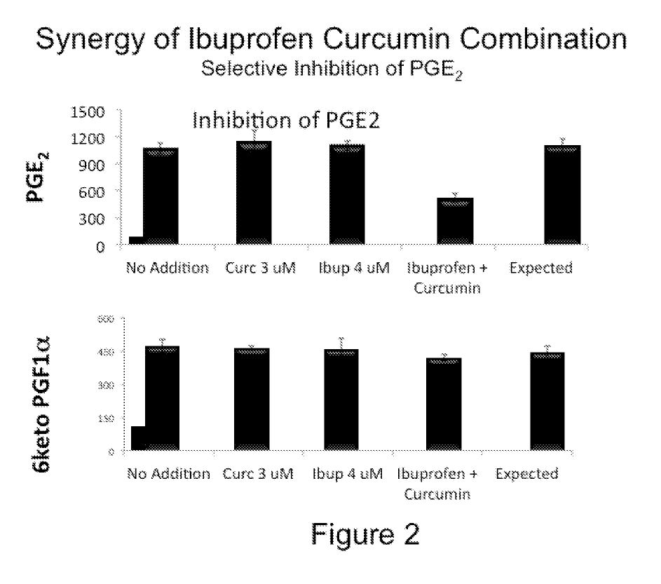

Synergism between ibuprofen and curcumin was demonstrated using the sensitization ratio method. Ibuprofen and curcumin alone and in 4:2 muM or 20:10 muM ratios (ibuprofen to curcumin) were coincubated with whole human blood stimulated with lipopolysaccharide (10 mug/ml) for 18 hours. Formation of two prostanoids PGE₂ and 6-keto PGF_{1α}, a stable metabolite of prostacyclin, a prostaglandin needed for cardiovascular function were determined by methods described earlier. Results after the determination of sensitization ratios (S.R.) are depicted in FIG. 2.

Curcumin (3 muM) and ibuprofen (4 muM) when added alone did not inhibit PGE₂ formation but in combination inhibited PGE₂ by about 50% showing a definite synergism with an S.R. of 12 (FIG. 2). Curcumin 10 muM showed no detectable inhibition of PGE2 but when combined with Ibuprofen 20 muM showed synergism as it improved its efficacy dramatically from 38% inhibition (alone) to 76% in combination with an S.R. of 2.0 (FIG. 2).

In the same aliquots of whole human blood assay the formation of 6-keto PGF_{1α} was also determined. In contrast with PGE₂, a combination of Curcumin (3 muM) and ibuprofen (4 muM) was not synergistic for the production of 6-keto PGF_{1α}. The observed and expected inhibition of 6-keto PGF_{1α} were very similar with S.R. of about 1.0 suggesting that the two inhibitors are acting independently (FIG. 2). A combination of Curcumin (10 muM) and ibuprofen (20 muM) demonstrated antagonism for the production of 6-keto PGF_{1α}. The observed inhibition was much less than expected inhibition of 6-keto PGF_{1α} with an S.R. of less than 1.0 (S. R.=0.76) suggesting that the two inhibitors are antagonistic for the production of 6-keto PGF_{1α}. (FIG. 2).

If synergistic interaction between curcumin and ibuprofen is mediated through mPGES-1 within prostanoid biosynthesis, the combination should only affect the formation of PGE₂ and should not interfere with the biosynthesis and release of other prostanoids including the formation of 6-keto PGF_{1α} (a stable metabolite of PGI₂). In fact, combination of curcumin and ibuprofen showed a definite synergism in the inhibition of PGE₂. In contrast, the inhibition of 6-keto PGF_{1α} was either independent or antagonistic, in LPS-stimulated whole blood (FIG. 1). Accordingly, the inventors concluded that the synergism between ibuprofen and curcumin is due to inhibition of mPGES-1 by curcumin.

### Example 4

### A Dose of Ibuprofen, Phosphatidylcholine and Curcumin was More Efficacious than a Dose of Ibuprofen and Curcumin in Healthy Human Volunteer 1

In a crossover study a healthy human volunteer received a single dose of ibuprofen 400 mg, or ibuprofen 100 mg, or 50 mg ibuprofen alone, or ibuprofen plus curcumin or ibuprofen, curcumin and PC; with a seven day washout period between dosing of each drug or drug combinations. Dosing compositions were prepared as described above. A blood sample (6 ml), immediately prior to dosing and at 105 minutes after dosing, was obtained as described above. Production of two prostanoids PGE₂, and 6-keto PGF_{1α} after LPS stimulation and plasma concentrations of ibuprofen were determined as described above and are shown in Table 1.

US Patent Application #20070093457, disclosed a method of reducing a dosage size of an at least one NSAID in the treatment of cancer and inflammatory diseases, comprising an effective amount of curcumin and an effective amount of at least one NSAID. Dosing with ibuprofen 50 mg alone or ibuprofen 50 mg plus curcumin produced a very similar inhibition of the release of PGE₂, a main mediator of fever, pain, and inflammation in LPS-stimulated whole blood from this volunteer (Table 1). No improvement in efficacy of ibuprofen 50 mg alone vs ibuprofen plus curcumin was observed. Plasma concentrations of ibuprofen were also not significantly different for dosing with ibuprofen 50 mg alone or ibuprofen 50 mg plus curcumin (Table 1). In contrast, dosing with a combination of ibuprofen, curcumin and phosphatidylcholine enhanced the inhibition of production of PGE₂, in LPS-stimulated whole blood from this volunteer was substantially enhanced (80% vs. 56% for ibuprofen alone; Table 1). Plasma concentrations of ibuprofen were not significantly different for dosing with ibuprofen 50 mg alone or ibuprofen 50 mg, curcumin and phosphatidylcholine combination (Table 1).

In the same aliquots of whole blood from this volunteer the formation of 6-keto PGF_{1α} was also determined. Dosing with ibuprofen plus curcumin or ibuprofen, curcumin and PC did not enhance the inhibition of 6-keto PGF_{1α} compared to dosing with ibuprofen alone in LPS-stimulated whole blood. Lack of increased inhibition of this prostanoid could benefit patients and result in reduced risk of heart attack, stroke, systemic and pulmonary hypertension, congestive heart failure and sudden cardiac death.

TABLE 1
Inhibition of Prostanoid Formation in Human Volunteer 1

| | | 6-keto | Plasma |
|---|---|---|---|
| | PGE₂ | PGF_{1α} | ibuprofen |
| Drug(s) Dose | (% inhibition) | (% inhibition) | (ug/ml) |
| Ibuprofen, 50 mg | 56 (3) | 61 (6) | 1.70 (0.15) |
| Ibuprofen, Curcumin, | 51 (5) | 54 (3) | 1.97 (0.18) |
| 200 mg | | | |
| Ibuprofen, Curcumin | 80 (4) | 47 (4) | 1.40 (0.17) |
| Phosphatidylcholine | | | |
| 400 mg | | | |

a. Number in parentheses is the standard error of 3 independent measurements

### Example 5

### A Dose of Ibuprofen, Phosphatidylcholine and Curcumin was More Efficacious than a Dose of Ibuprofen and Curcumin in Healthy Human Volunteer 2

In a 3-way crossover study, a healthy human volunteer received a single dose of 40 mg of ibuprofen alone, or ibuprofen plus curcumin or ibuprofen, curcumin and PC; with a seven day washout period between the dosing of each drug or drug combinations. Dosing compositions were prepared as described above. A blood sample (6 ml), immediately prior to dosing and at 105 min after dosing was obtained as described above. Production of two prostanoids PGE₂, and 6-keto PGF_{1α} after LPS stimulation and plasma concentrations of ibuprofen were determined as described above and are shown in Table 2.

Dosing with 40 mg of ibuprofen alone produced a 29% inhibition of the release of PGE₂, a main mediator of fever, pain, and inflammation in LPS-stimulated whole blood from this volunteer (Table 2). A slight improvement in inhibition of PGE₂ (34%) after ibuprofen plus curcumin dosing vs ibuprofen alone (29%) was observed. Plasma concentrations of ibuprofen were not significantly different for dosing with ibuprofen alone or ibuprofen plus curcumin (Table 2). A combination of ibuprofen, curcumin and phosphatidylcholine, however, was the most effective dose the inhibition of production of PGE₂, in LPS-stimulated whole blood 43% vs 29% for ibuprofen alone (Table 2). It is noteworthy that this enhancement of efficacy was observed at much lower plasma concentrations of ibuprofen after dosing with ibuprofen, curcumin and phosphatidylcholine combination (0.27ug/ml) than dosing with ibuprofen alone (0.42ug/ml; Table 2).

In the same aliquots of whole blood from this volunteer the formation of 6-keto PGF_{1α} was also determined. A slightly reduced inhibition of 6-keto PGF_{1α} after dosing with ibuprofen plus curcumin (24%) or ibuprofen, curcumin and PC (21%) compared to dosing with ibuprofen alone (35%) in LPS-stimulated whole blood was observed (Table 2). Lack of increased inhibition of this prostanoid could benefit patients and result in reduced risk of heart attack, stroke, systemic and pulmonary hypertension, congestive heart failure and sudden cardiac death.

TABLE 2
Inhibition of Prostanoid Formation in Human Volunteer 2

| | | 6-keto | Plasma |
|---|---|---|---|
| | PGE₂ | PGF_{1α} | ibuprofen |
| Drug(s) Dose | (% inhibition) | (% inhibition) | (ug/ml) |
| Ibuprofen, 40 mg | 29 (4) | 35 (2) | 0.42 (0.06) |
| Ibuprofen, | 34 (3) | 24 (6) | 0.32 (0.02) |
| Curcumin, 200 mg | | | |
| Ibuprofen, Curcumin | 43 (4) | 21 (3) | 0.27 (0.03) |
| Phosphatidylcholine | | | |
| 400 mg | | | |

a. Number in parentheses is the standard error of 3 independent measurements.

### Example 6

### Dosing with Ibuprofen, PC and Curcumin Enhanced the Inhibition of PGE_{2;} the Effect was Better than Ibuprofen Alone Administered at 2-Times Higher Concentrations

After undergoing a 3-way crossover study (Table 1) the same volunteer 1 also received two more higher doses of ibuprofen 100 mg and 400 mg; with a seven day washout period between dosing of each drug or drug combinations. Production of PGE₂ after LPS stimulation and plasma concentrations of ibuprofen were determined as described above and are shown in Table 3. For comparison purposes some of the data from Table 1 on dosing with ibuprofen 50 mg alone and ibuprofen 50 mg plus curcumin and phosphatidylcholine was also included in Table 3.

Inhibition of PGE₂ formation in human volunteer 1 is dose dependent with about 90% inhibition at a single dose of 400 mg of ibuprofen and about 50% inhibition at a dose of 50 mg ibuprofen (Table 3). When curcumin and phosphatidylcholine were included with a dose of 50 mg ibuprofen, it enhanced the efficacy of ibuprofen significantly (80% inhibition of PGE₂). Although the plasma concentrations of ibuprofen after dosing with PC, curcumin and ibuprofen 50 mg (1.4 ug/ml) were 2-times lower than that observed after administration of 100 mg ibuprofen alone (3.0 ug/ml). Significantly more inhibition of PGE₂ (80%) at a lower dose of ibuprofen (50 mg) in combination with PC and curcumin compared to dosing with higher concentration of ibuprofen 100 mg alone (71%) was observed (Table 3). The present inventors concluded that dosing with Ibuprofen, PC and curcumin enhances the inhibition of PGE₂ and the effect is better than administering the ibuprofen alone at 2-times the higher concentrations.

TABLE 3
Inhibition of PGE₂ Production in Human Volunteer 1

| | | Plasma |
|---|---|---|
| | PGE₂ | ibuprofen |
| Drug(s) Dose | (% inhibition) | (ug/ml) |
| Ibuprofen 400 mg | 88 (2) | 44.0 (3.0) |
| Ibuprofen 100 mg | 71 (5) | 3.0 (0.5) |
| Ibuprofen, 50 mg^{b} | 56 (3) | 1.7 (0.15) |
| Ibuprofen 50 mg, Curcumin | 80 (4) | 1.4 (0.17) |
| 200 mg, PC 400 mg^{b} | | |

a. Number in parentheses is the standard error of 3 independent measurements
^{b}Data from Table 1 and Example 3 included for comparison purposes

### Example 7

Dosing with Ibuprofen increases expression of pro-inflammatory cytokine TNF-α in human volunteers; inclusion of curcumin and PC can suppress this deleterious increase of the pro-inflammatory mediators.

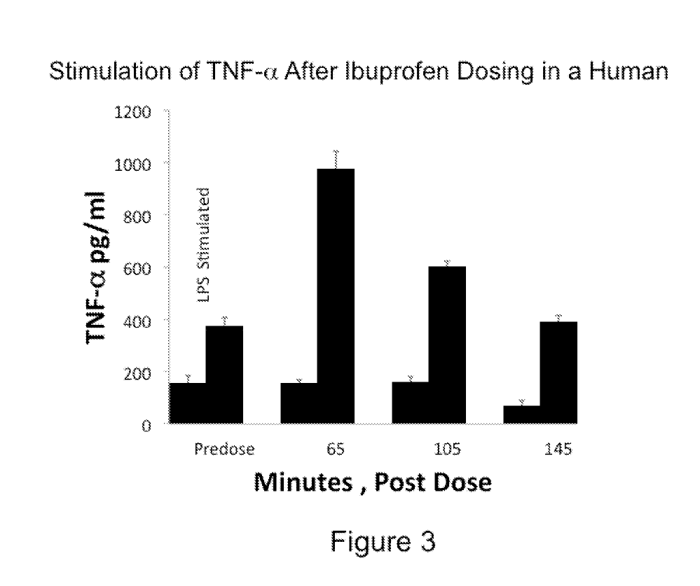

It has been taught that after surgical extraction of impacted third molars, treatment with rofecoxib and ibuprofen increases the expression of many pro-inflammatory mediators including inflammatory cytokines. Present inventors have also observed the deleterious increase of an inflammatory cytokine TNF-α after dosing with ibuprofen in human volunteers. In a study, volunteer 1 was given a single oral dose of 400 mg ibuprofen. A blood sample was collected into a tube containing heparin immediately prior to ibuprofen dosing and at the following times thereafter: 65, 105, and 115 mins. after dosing. An aliquot of blood was used for determination of TNF-α after LPS stimulation as described above. Data are shown in FIG. 3. A 3.5-fold increase in TNF-α 65 minutes after ibuprofen dosing was observed and the increase was reduced in blood samples collected after 65 minutes (FIG. 3).

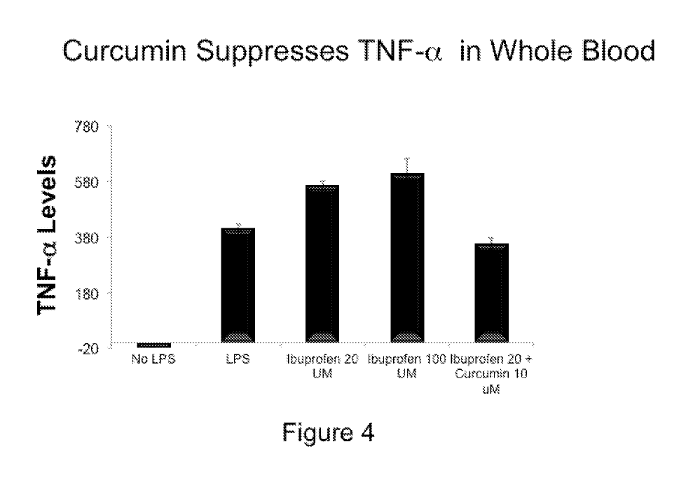

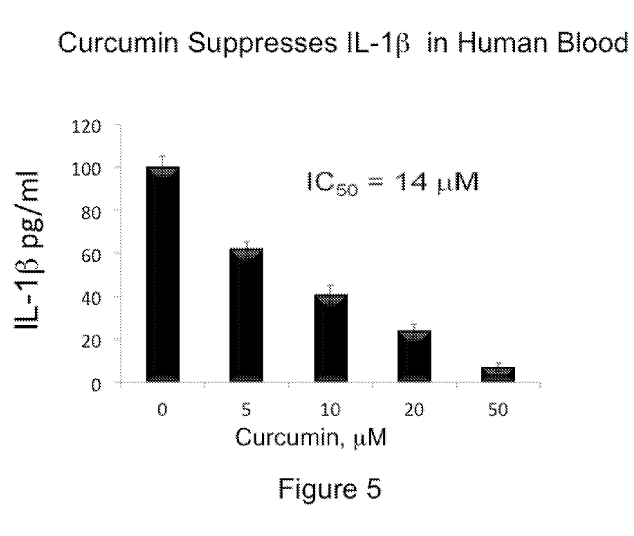

It is well known that treatment of OA patients with a combination of phospholipids from soy and curcumin significantly reduces levels of many inflammatory markers (interleukin [IL]-1 beta, IL-6, soluble CD40 ligand [sCD40L], soluble vascular cell adhesion molecule (sVCAM)-1, and erythrocyte sedimentation rate [ESR]) [Belcaro G. et al. Altern Med Rev. 2010; 15(4):337-44]. The blood from healthy adult volunteers was incubated with the indicated compounds for 5 mins. at room temperature; and then stimulated with LPS and inflammatory Cytokines (IL-1β& TNF-α) were determined as described above. Ibuprofen at 20 muM and 100 muM alone and in combination (ibuprofen 20 muM to curcumin 10 muM) were co-incubated with whole human blood stimulated with LPS. A dose-dependent stimulation of TNF-α by ibuprofen was observed and curcumin (10 muM) suppressed the deleterious stimulation of TNF-α (FIG. 4). A dose dependent suppression of IL-1 beta by curcumin with an IC₅₀=14 muM was also observed in another study (FIG. 5). The present inventors believe that synergistic combination ibuprofen, curcumin and PC will prevent local NSAID-induced increase in production of inflammatory cytokines that is expected to exacerbate synovial inflammation and degradation of cartilage in the arthritic joints. Suppression of inflammatory cytokines by this synergistic combination can also reduce the risks for cardiovascular heart disease (CHD) in patients.

In one embodiment, there are compositions and a method of treating a mammal with a composition which provides analgesia, antipyretic activity and anti-inflammatory effects in a patient in need thereof which includes administering a therapeutically effective amount of at least one dual inhibitor of mPGES-1 and NF-κB, at least one zwitterionic phospholipid and at least one NSAID.

The present invention arises from the discovery that a combination of at least one dual inhibitor of mPGES-1 and NF-κB, at least one zwitterionic phospholipids and at least one NSAID to be more effective than either therapy by itself. This observation can lead to the use of a smaller dosage of each compound, and in particular smaller amounts of toxic NSAID, thereby improving the safety profile of the NSAID regimen. In other embodiments larger doses can be administered without an increase in negative side effects of the NSAID(s) administered by the present method.

Thus, in another aspect of the present invention, there is provided a method for reducing a therapeutic dose size of an at least one NSAID in the treatment of a patient in need of an NSAID therapy, comprising simultaneous or step-wise administration of a dual inhibitor of mPGES-1 and NF-κB and at least one NSAID, the dual inhibitor of mPGES-1 and NF-κB being in an amount sufficient to reduce the NSAID concentration needed while maintaining the same therapeutic effect as compared to administering the NSAID alone.

In another aspect of the present invention, there is provided a method, wherein a dose of at least one NSAID is reduced to a safer lower dose from a higher dose and approved for over-the-counter (OTC) sale in the USA while maintaining the same therapeutic effect as compared to administering the NSAID alone at the higher dose for the treatment of a mammal in need of an NSAID therapy.

The preferred reduced therapeutic dose is one which improves therapeutic benefits of an NSAID but reduces short or long term adverse effects from use of smaller doses such as gastroduodenal ulcers, strictures, esophagitis, gastritis, colitis, small and large bowel erosions, acute and/or chronic renal failure, fluid and electrolyte imbalances, hyperflalemia, hematuria, nephrotic syndrome with interstitial nephritis, papillary necrosis, exacerbation of hypertension, exacerbation of congestive heart failure, arrhythmia, elevated transaminases, choleostasis, hepatic failure, headache, tinnitus, vertigo, tremor, depression, somnalence, altered mental status, aseptic meningitis, thrombocytopenia, hemolytic anemia, agranulocytosis, leukopenia, eosinophilia, aplastic anemia, exacerbation of asthma, cough, respiratory depression, laryngeal and pharyngeal edema, skin rash, photosensitivity, Stevens Johnson syndrome, pemphigoid reaction, erythema multiform, urticaria, angioedema, joint erosions or decreased repair of cartilage damage.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like, apparent to those skilled in the art still fall within the scope of the invention as claimed by the applicant.

What is claimed is:

1. A composition for improving the analgesic, antipyretic and anti-inflammatory effects of a non-steroidal anti-inflammatory drug (NSAID) in a mammal in need of NSAID therapy comprising:
a) at least one dual inhibitor of microsomal prostaglandin E₂ synthase-1 (mpges-1) and NF-κB;
b) at least one zwitterionic phospholipid; and
c) a therapeutically effective amount of one or more NSAID.

2. The composition according to claim 1 wherein NSAID is selected from the group consisting of, fenoprofen calcium, flurbiprofen, suprofen, benoxaprofen, Ibuprofen, ketoprofen, naproxen, oxaprozin, diclofenac sodium, diclofenac potassium, aceclofenac, etodolac, indoniethacin, ketorolac tromethamine, ketorolac, nabumetone, sulindac, tolmetin sodium, meclofenamate sodium, metenamic acid, piroxicam, lomoxicam and meloxicam, diflunisal, aspirin, magnesium salicylate, bismuth subsalicylate, oxyphenbutazone, phenylbutazone, celecoxib, rofecoxib, valdecoxib.

3. The composition according to claim 1, wherein the dual mPGES-1 and NF-κB inhibitor is selected from the group consisting of, curcumin, epi-gallocatechin gallate, garcinol, myrtucommulone, arzanol, β-boswellic acid, keto-β-boswellic acid, acetyl-keto-β-boswellic acid (AKBA), 2-(2-hydroxy-4-methoxyphenyl)-5-(3-hydroxypropyl)benzofuran, (+)-conocarpan, carnosol, carnosic acid, physodic acid, imbricaric acid and perlatolic acid, Embelin (2,5-dihydroxy-3-undecyl-1,4-benzoquinone), and acylphloroglucinol hyperforin.

4. The composition according to claim 3, wherein the dual mPGES-1 and NF-κB inhibitor is selected from the group consisting of: pure curcumin and a mixture of at least curcumin, demethoxycurcumin and bisdemethoxycurcumin.

5. The composition according to claim 3, wherein the dual mPGES-1 and NF-κB inhibitor is selected from the group consisting of at least one of pure beta-Boswellic Acid, alpha-Boswellic Acid and beta-Boswellic Acid.

6. The composition according to claim 1, wherein the at least one zwitterionic phospholipids is selected from the group consisting of: phosphatidyl choline (PC), dipalmitoylphosphatidylcholine (DPPC), disaturated phosphatidylcholines, phosphatidylethanolamines, phosphatidylinositol, phosphatidylserines sphingomyelin, lecithin oils derived from soy beans, dimyristoylphosphatidylcholine, distearoylphosphatidylcholine, dilinoleoylphosphatidylcholine, dipalmitoylphosphatidylcholine, soy phophatidylchloine and egg phosphatidycholine and the neutral lipid is tripalmitin.

7. A method of treating a mammal in need of at least one of an analgesic, antipyretic and anti-inflammatory treatment using a (NSAID) in a mammal in need of NSAID therapy comprising administering to the mammal and effective amount of a composition comprising:
a) at least one dual inhibitor of microsomal prostaglandin E₂ synthase-1 (mpges-1) and NF-κB;
b) at least one zwitterionic phospholipid; and
c) a therapeutically effective amount of one or more NSAID.
